(19) United States
(12) Patent Application Publication
(10) Pub. No.: US 2010/0168001 A1
(43) Pub. Date: Jul. 1, 2010

(54) Method for Preventing and/or Treating Articles or Media Contaminated With Bacteria of the Legionelle Genus

(76) Inventors: Yann Hechard, Valdivienne (FR); Jean-Marc Berjeaud, Savigny I'Evescault (FR)

Correspondence Address: NIXON & VANDERHYE, PC, 901 NORTH GLEBE ROAD, 11TH FLOOR, ARLINGTON, VA, 22203, US

(21) Appl. No.: 12/086,915

(22) PCT Filed: Dec. 19, 2006
(86) PCT No.: PCT/FR2006/002786

§ 371 Date: Sep. 9, 2008

(30) Foreign Application Priority Data
Dec. 20, 2005 (FR) 05/12955

(51) Int. Cl. A61K 38/16; C07K 14/00

(52) U.S. Cl. 514/12; 530/326; 530/324; 514/13

(57) ABSTRACT

The present invention relates to peptides that are active against bacteria of the Legionella genus, characterized in that they correspond to the amino acid sequence corresponding to SEQ ID No. 1: MQFITDLIKKAVDFFKGLFGNK, or to any other similar sequences. The invention also relates to the use of such peptides for preventing the contamination of surfaces or of materials with bacteria of the Legionella genus or for decontaminating surfaces or materials infected with Legionella.

The present invention relates to a method for preventing and/or treating articles, materials or surfaces, or media contaminated with bacteria of the Legionella genus. Its subject is also the compositions used and the peptides that are active against these bacteria, called hereinafter “anti-Legionella peptides”. Bacteria of the Legionella genus belong to the Legionellaceae family. The Legionella genus is divided into 48 species comprising 70 distinct serogroups. The species Legionella pneumophila (L. pneumophila) is the bacteria responsible for legionellosis, a fatal pneumonia in about 15% of cases. This bacterium is mainly present in fresh and lukewarm water.

In water pipes, Legionella is present in the biofilms where it is capable of surviving. Biofilms are microbial communities that adhere to a surface and are frequently contained in a matrix of extracellular polymers. Biofilms are present not only in nature but also in the human environment (pipelines, medical apparatus and the like). Legionella can also be found in the free state or associated with amoebae, in which it develops. Amoebae are unicellular eukaryotes which belong to the protozoa family. They play a crucial role in the amplification of Legionella populations in aquatic systems and they protect them from the unfavorable external conditions. They also provide them with a particular environment which will promote their virulence to all human cells.

The infection of humans with Legionella occurs exclusively by inhalation of contaminated droplets of water or of contaminated aerosols induced by various sources such as wet cooling systems (cooling towers, air conditioning systems and the like), showers and the like. L. pneumophila multiplies in tap water at temperatures of between 25 and 45° C., with an optimum growth temperature of the order of 35° C. On the other hand, L. pneumophila develops in a pH range of between 5.5 and 9.2, with an optimum growth pH equal to 6.9.

Various treatments may be used to limit the development of Legionella in water. Thermal disinfection (≧70° C.) may be used, but the most common treatments are chemical treatments (chlorine and derivatives, treatments with oxidants, metal ions and the like) (Kim et al. (2002), Water Res 36, 4433-4444). However, these treatments are not specifically targeted against Legionella, which has the effect in particular of disrupting the ecological balance of all the microorganisms in the environment. In addition, as soon as the treatment ends, Legionella develops again, showing the limited efficiency of the process. Finally, the chemical products used are a major source of environmental pollution.

The publication Héchard et al. (2005, FEMS Microbiol. Lett., 252, 19-23) discloses that the Staphylococcus warneri (S. warneri) strain inhibits the development of Legionella by virtue of a molecule secreted by S. warneri which appears to be a bacteriocin.

Bacteriocins are antibacterial proteins or peptides produced by bacteria. They are produced by both Gram-negative bacteria and Gram-positive bacteria. They were first described in E. coli and named colicins. In Gram-positive bacteria, the bacteriocins produced by lactic acid bacteria are the ones that have been most widely studied given their potential use in the agri-foodstuffs sector and more rarely in hygiene or health.

U.S. Pat. No. 5,817,362 describes a bacteriocin produced by Lactococcus lactis, which inhibits the development of a large number of bacteria such as Staphylococcus, Pediococcus, Lactococcus, Lactobacillus and the like, and which can be used in the food sector. U.S. Pat. No. 5,445,835 describes a method for producing a yogurt comprising the bacteriocin PA-1. Patent EP 927023 relates to a composition for treating acne, comprising a bacteriocin produced by Propionibacterium. Patent EP 851751 relates to a chewing gum comprising a bacteriocin as antibacterial agent. Application WO01/92533 relates to a class IIa bacteriocin produced from a specific strain of Lactobacillus sakei, which is found to be particularly effective for inhibiting the growth of Listeria, and which can therefore be used industrially as active agent against pathogenic or undesirable flora in the preparation of food products.

Apart from the abovementioned publication (Héchard et al. 2005, FEMS Microbiol. Lett., 252, 19-23), no prior art document describes a bacteriocin having activity against Legionella. In the Héchard et al. publication, the bacteriocin is however described in an imprecise, incomplete and, in addition, erroneous manner because the authors did not succeed in purifying, isolating and identifying the molecule responsible for the activity against Legionella. Indeed, mass spectrophotometry analysis of the active anti-Legionella fraction reveals two molecular masses of 2613.8 Da and 2449.4 Da respectively. The purification work by the inventors on the peptides secreted by Staphylococcus and more especially by S. warneri allowed them to characterize peptides of great interest. Their study indeed showed a high activity of these peptides against Legionella and more specifically against Legionella pneumophila.

The aim of the invention is therefore the use of such peptides as anti-Legionella compounds.

Its aim is also to provide a method of decontamination specifically targeted against bacteria of the Legionella genus, taking advantage of the properties of the peptides isolated from compounds secreted by Staphylococcus and more especially S. warneri.

The invention also aims to provide a method of treatment for the decontamination of Legionella without disrupting the ecological balance of the environment.

The invention additionally relates to the use of the isolated active peptides for the manufacture of medicaments for the treatment of Legionella.

According to yet another aspect, the invention relates to a method of treating a patient infected with legionellae, using said peptides.

The invention therefore relates to the use of at least one peptide having an anti-Legionella effect for example in the test reported in the examples, the peptide(s) being isolated and purified from compounds secreted by Staphylococcus, and more especially by S. warneri.

These anti-Legionella peptides are advantageously chosen from the group comprising peptides having the amino acid sequence SEQ ID No. 1:

MQFITDLIKKAVDFFKGLFGNK,

and peptides of the family of delta-hemolysins synthesized from RNA III, in particular delta-hemolysin 1 and delta-hemolysin 2 isolated by purification from compounds secreted by S. warneri and corresponding, respectively, to the following sequences SEQ ID No. 2 and SEQ ID No. 3:

| | |
|---|---|
| SEQ ID No. 2: | MAADIISTIGDLVKLIINTVKKFQK, |
| SEQ ID No. 3: | MTADIISTIGDFVKWILDTVKKFTK. |

These peptides may also be substituted and have for example a formyl substitution, it being understood that their substitution does not significantly affect the anti-Legionella activity as evaluated in the examples.

The invention also relates to a method for the prevention of surfaces or materials liable to be in contact with bacteria of the Legionella genus and/or for the decontamination of surfaces or materials infected with Legionella, this method comprising bringing the surface or the material to be treated into contact with a composition containing an effective quantity of at least one peptide as defined above.

The compositions used in the above applications are advantageously in the form of solutions or suspensions and contain, where appropriate, adjuvants for facilitating the application and/or enhancing the activity of the peptides.

As a variant, they are pulverulent compositions to be used as they are or to be diluted at the time of use.

The invention also relates to the corresponding method of treatment which comprises the addition, in an appropriate form, of said peptides to air or water.

Advantageously, the peptides used according to the invention have activity against all the bacteria of the Legionella genus. By contrast, they are not active against the other non-Legionella bacteria.

These peptides are in particular active against bacteria of the Legionella genus chosen from the group consisting of the species Legionella pneumophila, Legionella bozemanii, Legionella dumofii, Legionella longbeachae, Legionella micdadei, Legionella feeleii, Legionella hackeliae, Legionella sainthelensi, Legionella spiritensis, Legionella erythra and Legionella quinlivanii.

According to yet another aspect, the subject of the present invention is the use of one or more peptides as defined above, for the preparation of a medicament intended for combating legionellosis.

In this use, the medicament contains a quantity of active ingredient which makes it possible to obtain the effect desired by the treatment and is provided in the appropriate forms for administration, for example for a treatment by the oral or parenteral route, or by inhalation.

For administration by the oral route, tablets, lozenges or gelatin capsules are used for example.

Forms suitable for administration by the parenteral route comprise sterile or sterilizable solutions or suspensions of said peptides.

For inhalation, aerosols are advantageously used.

The peptides having the sequence SEQ ID No. 1 are novel products and as such fall within the scope of protection of the invention. They comprise the peptide SEQ ID No. 1 and substituted peptides as indicated above.

The pharmaceutical compositions containing a therapeutically effective quantity of this peptide having the sequence SEQ ID No. 1 or novel anti-Legionella peptides as defined above in combination with a pharmaceutically inert vehicle are also covered by the invention.

The invention additionally relates to transfer or expression vectors, in particular of the plasmid type, characterized in that they contain at least one DNA fragment as defined above.

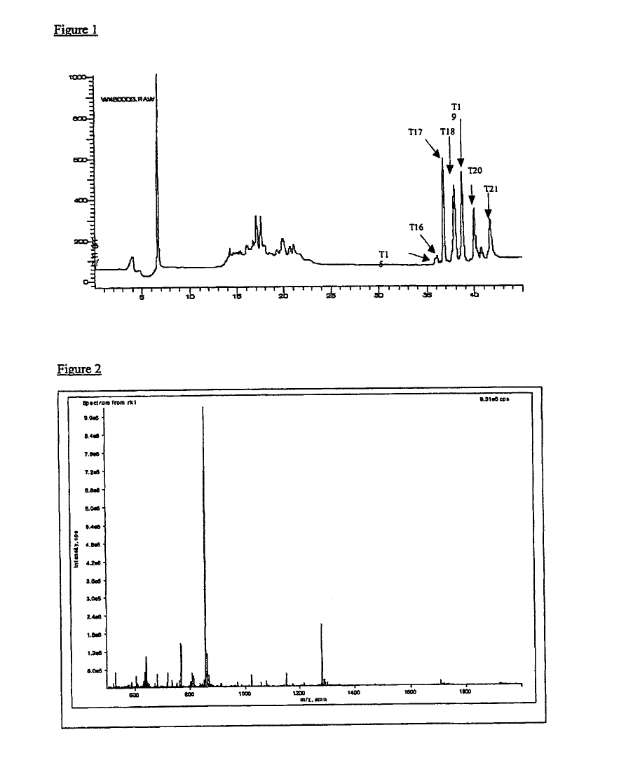

Other characteristics and advantages of the invention will be given in the examples which follow, in which reference is made to FIGS. 1 to 4, which represent, respectively:

FIG. 1, the chromatogram obtained during HPLC of the seven fractions T15 to T21 obtained by solid-phase extraction with an acetonitrile-TFA elution gradient starting with 20% acetonitrile,

FIG. 2, the mass spectrum of the active fraction T17,

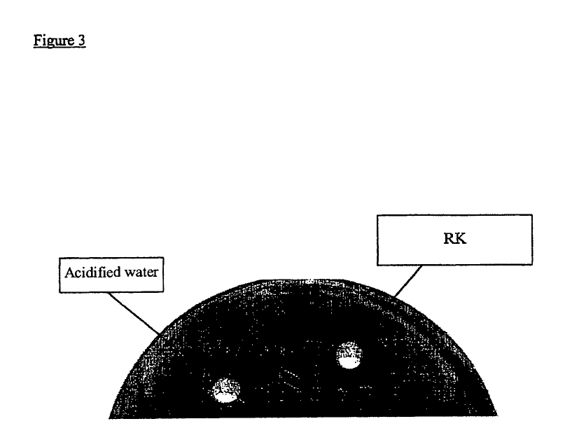

FIG. 3, the results of the activity test in wells obtained with the HPLC-purified fraction.

### EXAMPLE 1

### Isolation and Characterization of Peptides Active Against Legionella from the Staphylococcus warneri Strain

The Staphylococcus warneri bacterial strain, named “RK”, is isolated as indicated in the article by the inventors Héchard et al. (2005, FEMS Microbiol. Lett., 252, 19-23).

1) Purification of the Bacterial Strain

The anti-Legionella peptide SEQ ID No. 1 was purified by a method which differs from that described in the article by the inventors since it was not possible in the protocol described in Héchard et al. (2005, FEMS Microbiol. Lett., 252, 19-23) to obtain a pure peptide and since only a mixture was obtained.

This article did not therefore make it possible to foresee that the peptide of the invention obtained by purification existed and that it was then possible to obtain a product that has great efficacy since it can be industrially exploited because it is pure.

Among the numerous possibilities for changing purification protocol that can be envisaged by a person skilled in the art, modifications relating to (a) the percentage of trifluoroacetic acid used in the eluents which was reduced to 0.05%, and (b) the solubilization of the sample before injection into HPLC which was performed with water acidified to pH 5 with HCl instead of a mixture of water and acetonitrile.

The Staphylococcus warneri RK strain was cultured for 24 h in a BHI (Brain Heart Infusion) medium (200 ml in a 1 L Erlenmeyer with stirring at 37° C.). The culture is centrifuged (6000 g, 15 min, 4° C.). The supernatant is preserved and heated for 15 min at 70° C. This sample constitutes the crude extract.

The pH of the crude extract is brought to 5 with a hydrochloric acid HCl solution. The solution (100 ml) is then passed through a cation-exchange chromatography column (Hiprep™ 16/10 Carboxy-Methyl FF, Amersham Biosciences), successively washed with 100 ml of sodium acetate buffer (20 mM, pH 5) containing 0, 0.1 and 1 M NaCl. The growth of L. pneumophila is inhibited by the fraction eluted with sodium acetate containing 1 M NaCl.

This fraction (˜100 ml) is then loaded onto a solid-phase extraction cartridge (Sep-pak C18, Waters) previously washed and equilibrated (10 ml of ethanol, 5 ml of acetonitrile and 10 ml of Milli-Q water). The elution is performed sequentially with 5 ml of solution with an increasing concentration of acetonitrile (0, 20, 40, 80 and 100%) and containing 0.05% of trifluoroacetic acid (TFA). The various fractions harvested are then concentrated and freeze-dried and finally suspended in 1 ml of sterile water, and then tested for activity against L. pneumophila.

L. pneumophila is inhibited with the fraction containing 80% acetonitrile.

This fraction is injected into a Kromasil C8 analytical column (250×4 mm) and analyzed by HPLC (Perkin Elmer). The chromatogram obtained is represented in FIG. 1. This chromatogram is significantly different from that published in Héchard et al. (2005, FEMS Microbiol. Lett., 252, 19-23). Indeed, in the Héchard et al. publication, the active peptide is eluted as a mixture with other peptides which alone are detected by mass spectrometry.

In the present invention, the peptide is completely isolated. It exhibits great efficiency which can be industrially exploited since it is pure.

The elution is performed with a linear H₂O-acetonitrile-TFA (0.05%) gradient ranging from 20% to 100% acetonitrile over 45 min with the last 10 minutes at 100% acetonitrile-TFA (flow rate: 0.8 ml.min⁻¹). The detection is performed at 220 nm. After evaporation of the acetonitrile and freeze-drying, each of the fractions, T15 to T21, is suspended in 1 ml of sterile water and then tested for activity.

Test for Anti-Legionella Activity in Wells

A suspension of Legionella pneumophila Lens is prepared in 1 ml of sterile Milli-Q water. The optical density (OD) at 600 nm with 1/10th of the suspension is measured. A required volume of water is added to the remaining 900 μl of suspension in order to have at the end a solution having an OD equal to 1.

100 μl of this solution are deposited on a BCYE (Buffered Charcoal Yeast Extract) plate for spreading (which is performed with the aid of a spreader). The composition of the BCYE medium is the following:

| | | |
|---|---|---|
| ACES buffer | 5 | g |
| yeast extract | 10 | g |
| activated charcoal | 2 | g |
| agar | 15 | g |
| L-cystein | 0.4 | g |
| ferric pyrophosphate | 0.25 | g |
| water | qs 1 | l |

The wells are made in BCYE agar with the aid of a hollow punch.

100 μl of the test solution (T15 to T21) are deposited in a well. The plate is then incubated at 37° C. in the presence of 5% CO₂ for 72 h.

Fractions T17, T18 and T19 are active against Legionella.

Analysis by electrospray ionization mass spectrometry of the active fraction T17 made it possible to determine the mass of the corresponding peptide: 2561.04 Da (see FIG. 2).

The antagonistic effect is responsible for a Legionella inhibition zone around the wells whose radius is proportional to the concentration of bacteriocin.

FIG. 3 illustrates the anti-Legionella activity test in wells of the HPLC-purified fraction T17.

The characterization of fractions T18 and T19 showed that they were delta-hemolysins 1 and 2, respectively. Microsequencing of the isolated and purified peptides led to the sequences SEQ ID No. 2 and SEQ ID No. 3. Their respective molecular masses are 2760.00 Da (delta-lysin 1) and 2871.00 Da (delta-lysin 2).

2) Sequencing of the Anti-Legionella Peptide

Microsequencing of the purified peptide SEQ ID No. 1 of the invention was performed at the Centre Commun de Séquençage de 1'IBCP (IBCP Common Sequencing Center) in Lyons.

The theoretical calculation of the mass of the complete sequence SEQ ID No. 1 leads to 2561.08 Da.

Advantageously, the peptide having the sequence SEQ ID No. 1 was also obtained by the peptide synthesis route (Eurogentec). The synthetic peptide has the same properties as the peptide obtained from the bacterial strain: same retention time in HPLC, same mass spectrum and identical activity.

### EXAMPLE 2

### Isolation and Characterization of the Delta-Hemolysins 1 and 2 from the S. warneri Strain

The procedure is carried out as indicated above, but applying an acetonitrile gradient starting at 50%.

1. A method for the prevention and/or treatment of articles, such as materials and surfaces, or of media such as water or air, against contamination with Legionella, characterized in that it comprises bringing the surface or the material to be treated into contact with a composition containing an effective quantity of at least one peptide having an anti-Legionella effect isolated and purified from compounds secreted by Staphylococcus, and more especially by S. warneri.

2. The method as claimed in claim 1, characterized in that the peptides are chosen from the group comprising peptides having the amino acid sequence SEQ ID No. 1:
MQFITDLIKKAVDFFKGLFGNK,
or peptides of the family of delta-hemolysins synthesized from RNA III.

3. The method as claimed in claim 2, characterized in that the hemolytic peptides consist of delta-hemolysin 1 and delta-hemolysin 2 and correspond, respectively, to the following sequences SEQ ID No. 2 and SEQ ID No. 3:

| | |
|---|---|
| SEQ ID No. 2: | MAADIISTIGDLVKLIINTVKKFQK, |
| SEQ ID No. 3: | MTADIISTIGDFVKWILDTVKKFTK. |

4. The method as claimed in claim 1, characterized in that the compositions used are in the form of solutions or suspensions and contain, where appropriate, adjuvants for facilitating the application and/or enhancing the activity of the peptides.

5. The method as claimed in claim 1, characterized in that the compositions used are in pulverulent form and are used as they are or diluted at the time of use.

6. The method as claimed in claim 1, characterized in that it is used to combat bacteria of the Legionella genus chosen from the group consisting of the species Legionella pneumophila, Legionella bozemanii, Legionella dumofii, Legionella longbeachae, Legionella micdadei, Legionella feeleii, Legionella hackeliae, Legionella sainthelensi, Legionella spiritensis, Legionella erythra and Legionella quinlivanii.

7. A peptide that is active against bacteria of the Legionella genus, characterized in that it corresponds to the amino acid sequence SEQ ID No. 1:
MQFITDLIKKAVDFFKGLFGNK,
and corresponding substituted peptides.

8. The use of at least one peptide as used in the method as claimed in claim 1, for the preparation of a medicament intended for combating legionellosis.

9. A pharmaceutical composition, characterized in that it contains a therapeutically effective quantity of a peptide having the sequence SEQ ID No. 1 in combination with a pharmaceutically inert vehicle.


SEQUENCE LISTING (3 sequences)

SEQ ID NO: 1 - protein, 22 aa, Artificial Sequence
  Description of Artificial Sequence Synthetic peptide
MQFITDLIKKAVDFFKGLFGNK

SEQ ID NO: 2 - protein, 25 aa, Artificial Sequence
  Description of Artificial Sequence Synthetic peptide
MAADIISTIGDLVKLIINTVKKFQK

SEQ ID NO: 3 - protein, 25 aa, Artificial Sequence
  Description of Artificial Sequence Synthetic peptide
MTADIISTIGDFVKWILDTVKKFTK